(19) United States
(12) Patent Application Publication
(10) Pub. No.: US 2025/0134723 A1
(43) Pub. Date: May 1, 2025

(54) METHODS OF MANUFACTURING AND USING RADIOPAQUE LOCALIZATION ARRAYS

(71) Applicant: MinRay, LLC, Wake Forest, NC (US)

(72) Inventors: Jeffrey Daniel Forbes, Wake Forest, NC (US); Matthew Bartlett Hedrick, Carmel, IN (US)

(73) Assignee: MinRay, LLC, Wake Forest, NC (US)

(21) Appl. No.: 18/497,926

(22) Filed: Oct. 30, 2023

(51) Int. Cl. A61F 13/02; C09J 7/25

(52) U.S. Cl. CPC A61F 13/024; A61F 13/0253; C09J 7/255; C09J 2301/502; C09J 2467/006

(57) ABSTRACT

Methods of manufacturing a radiopaque localization array are provided. A radiopaque layer and a backing layer may be provided. The radiopaque layer and the backing layer may be combined with an adhesive layer between the radiopaque layer and the backing layer. The radiopaque localization array may be cut in the radiopaque layer by a cutter.

### TECHNICAL FIELD

This application relates to the medical devices and, in particular, to radiopaque localization arrays.

### BACKGROUND

Present methods of manufacturing radiopaque localization arrays suffer from a variety of drawbacks, limitations, and disadvantages. Accordingly, there is a need for inventive methods and inventive radiopaque localization arrays described herein.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates an example of a radiopaque localization array cut from a metal foil;

FIG. 2 illustrates an example of a method of manufacturing the radiopaque localization array by cutting the radiopaque localization array in a radiopaque layer using a cutter;

FIG. 3 illustrates an example of a method of manufacturing the radiopaque localization array by cutting the radiopaque localization array in a radiopaque layer before combining the radiopaque layer and the backing layer;

FIG. 4 illustrates an example of a method of manufacturing the radiopaque localization array by cutting the radiopaque localization array in a radiopaque layer when combining the radiopaque layer and the backing layer; and

FIG. 5 illustrates an example of a method of using the radiopaque localization array that leverages direct access to the surface of the patient.

### DETAILED DESCRIPTION

In one example, a method of manufacturing a radiopaque localization array is provided. The radiopaque localization array may be any structure that is opaque to electromagnetic radiation emitted by a medical imaging machine, and is configured to identify a location on the skin of a patient relative to an internal feature of the patient in an image taken by a medical imaging machine. In the example of the method of manufacturing, a radiopaque layer, an adhesive layer, and a backing layer are provided. The radiopaque layer and the backing layer are combined with an adhesive layer between the radiopaque layer and the backing layer. The radiopaque localization array is then cut in the radiopaque layer by a cutter.

One technical advantage of some of the methods described herein is that radiopaque localization arrays may be manufactured at lower costs than with some other methods.

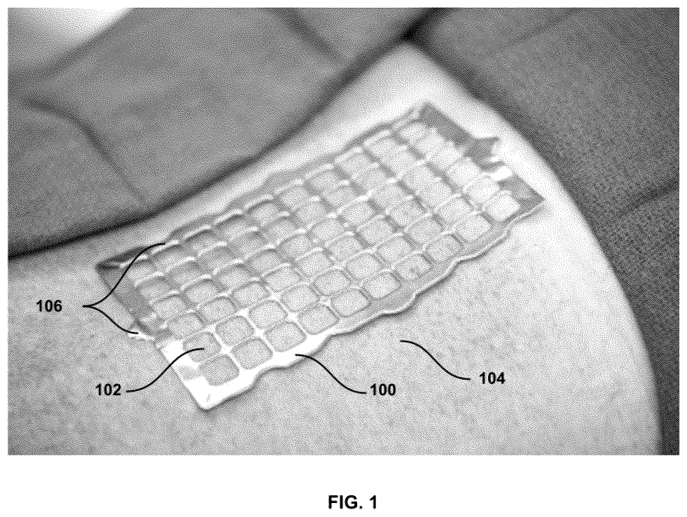

FIG. 1 illustrates an example of a radiopaque localization array 100 cut from a metal foil. The radiopaque localization array 100 may be any structure that is opaque to electromagnetic radiation emitted by a medical imaging machine (not shown), and is configured to identify a location 102 on a surface 104 of a patient relative to an internal feature (not shown) of the patient in an image taken by the medical imaging machine. Examples of the medical imaging machine include an x-ray machine and a CT scanner. Examples of the internal feature may be a gap between bones, a joint, a bone, cartilage, a tumor, a portion of the patient's anatomy, or any other feature identifiable in an image taken by the medical imaging machine.

The radiopaque localization array 100 may be configured to identify the location 102 relative to the internal feature shown in the image by virtue of one or more markings 106. Only a subset of the markings 106 of the radiopaque localization array 100 are identified with callouts in FIG. 1 in order to avoid cluttering the figure with callouts. Each of the markings 106 may be any structure that may identify a location on the radiopaque localization array 100. The markings 106 may include, for example, indentations, projections, openings, symbols, letters, numbers, and/or any other shape that may enable identification of the location 102 within or around the radiopaque localization array 100. Because the radiopaque localization array 100 has portions that are radiopaque and portions that are radio transparent, the radiopaque localization array 100 will appear superimposed on one or more internal features of the patient in the image taken by the medical imaging machine. One or more of the markings 106 visible in the image may identify where one or more internal features visible in the image are relative to the markings 106. Correspondingly, the markings 106 of the radiopaque localization array 100 may effectively identify where the internal features are relative to the surface 104 of the patient. For example, one of the markings 106 visible in the image that is adjacent to an internal feature visible in the image may be considered to correspond to the location 102 on the surface 104 of the patient that is adjacent to the internal feature. Thus, the radiopaque localization array 100 may aid in, for example, verifying a proper incision site, and/or a surgical approach angle in relation to the internal feature. Openings in the radiopaque localization array 100 may enable the surface 104 of the patient to be seen and/or physically accessed by a health care practitioner.

In the illustrated example, the radiopaque localization array 100 is a rectangular grid—specifically, a metal grid—having regularly spaced openings. However, the radiopaque localization array 100 may be in any form that includes one or more of the markings 106. For example, the radiopaque localization array 100 may be circular having openings arranged in a radial pattern.

The portions of the radiopaque localization array 100 that are radiopaque need to be distinguishable in the image from the portions of the radiopaque localization array 100 that are radio transparent. Accordingly, the portions that are radiopaque do not necessarily need to be fully opaque and the portions that are radio transparent do not necessarily need to be fully transparent.

With respect to identifying the location 102 on the surface 104 of the patient with the radiopaque location array 100, the location 102 identified may be any point or area of the surface 104 of the patient. The surface 104 may be any exposed part of the patient to which the radiopaque location array 100 may be placed. For example, the surface 104 may be skin or any other exposed tissue.

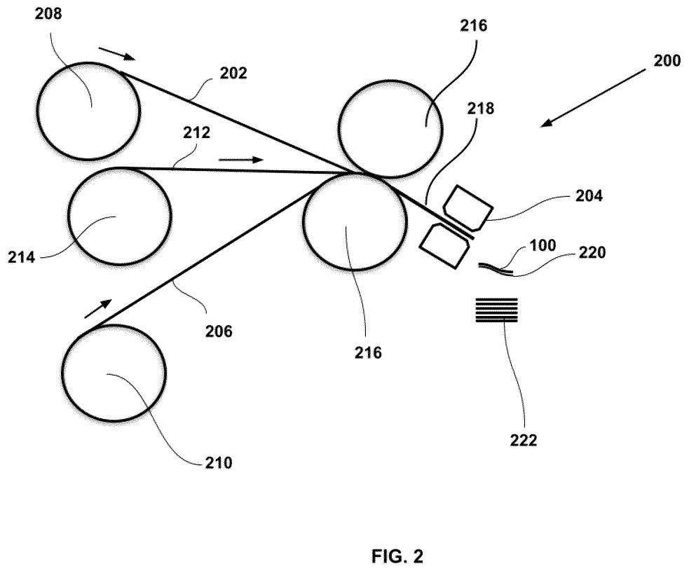

FIG. 2 illustrates an example of a method 200 of manufacturing the radiopaque localization array 100 by cutting the radiopaque localization array 100 in a radiopaque layer 202 using a cutter 204. In the illustrated example, the radiopaque localization array 100 has a removable backing 220 derived from a backing layer 206 as explained further below.

The radiopaque layer 202 may be provided by turning a radiopaque layer spool 208 on which the radiopaque layer 202 was initially wrapped. The backing layer 206 may be provided by turning a backing layer spool 210 on which the backing layer 206 was initially wrapped. An adhesive layer 212 may be provided by turning an adhesive layer spool 214 on which the adhesive layer 212 was initially wrapped.

The radiopaque layer 202 may comprise any material that is opaque (completely or partially) to electromagnetic radiation emitted by the medical imaging machine. The opaque material may be distributed uniformly or substantially uniformly across or within the radiopaque layer 202. In any case, the opaque material is not distributed within the radiopaque layer 202 in shape of the radiopaque localization array 100, thus requiring the cutting of the radiopaque localization array 100 in a radiopaque layer 202. The opaque material may include, or be, a metal layer. The opaque material may include a metal foil, a metal film, metal strips, wire or wires, metal particles, a metal coating, and/or a chemical compound comprising a metal. In some examples, the metal foil may be a 5 mil copper foil and/or a 5 mil copper sheet.

The backing layer 206 may comprise any material that prevents the adhesive layer 212 from inadvertently adhering to anything other than a target surface, such as the surface 104 of the patient. Alternatively, or in addition, the backing layer 206 may be any material that provides structural support to the radiopaque layer 202. Alternatively, or in addition, the backing layer 206 may comprise any material that provides anti-slip properties. The backing layer 206 may include any suitable material, for example, paper, a plastic film, a polyethylene terephthalate film, cloth, foam, rubber, and/or metal foil.

The adhesive layer 212 may include any suitable adhesive material, such as a two-sided adhesive polymer. The polymer in the adhesive layer 212 may be rigid or flexible. The polymer may, in some examples, comprise a rigid panel, strips, and/or wire(s). Alternatively, or in addition, the polymer may be flexible and be in any form such as a film, a coating, a fiber, yarn, fabric, non-woven fabric, and/or woven fabric. The polymer may include nano, submicron, micron, and/or larger fibers. The polymer may be any polymer, for example, polyester, polypropylene, polyurethanes, rubber, and nylon.

The radiopaque layer 202 and the backing layer 206 may be combined with the adhesive layer 212 between the radiopaque layer 202 and the backing layer 206. This may be accomplished, for example, by feeding the adhesive layer 212 between the radiopaque layer 202 and the backing layer 206. In the example shown in FIG. 2, the combining of the radiopaque layer 202 and the backing layer 206 includes bringing the radiopaque layer 202, the adhesive layer 212, and the backing layer 206 into contact with each other by two rollers 216 positioned adjacent to each other. A multilayer structure 218 comprising a section of the radiopaque layer 202 and a section of the backing layer 206 is formed as a result. The adhesive layer 212 or a section of the adhesive layer 212 is included in the multilayer structure 218 between the sections of the radiopaque layer 202 and the backing layer 206. The section of the radiopaque layer 202 and the section of the backing layer 206 are held together by the adhesive layer 212 or a section thereof. The adhesive selected for the adhesive layer 212 may be weak enough that the backing layer 206 may be later peeled off of the radiopaque layer 202 by hand, but strong enough to otherwise hold the multilayer structure 218 together. In some other examples, the adhesive selected for the adhesive layer 212 may be a permanent adhesive so that the backing layer 206 is not removable.

The radiopaque localization array 100 may be cut in the radiopaque layer 202 of the multilayer structure 218 using the cutter 204. The cutter 204 may be any cutting tool capable of removing one or more portions of the radiopaque layer 202 so that the radiopaque localization array 100 or a portion thereof is created. Examples of the cutter 204 include a die cut machine, a stamping press, and a laser.

The radiopaque localization array 100 may be attached to the removable backing 220 derived from the backing layer 206. For example, as the radiopaque localization array 100 is cut in the radiopaque layer 202 of the multilayer structure 218, the cutter 204 may also cut all the way through the backing layer 206 of the multilayer structure 218. As a result, in some examples, the removable backing 220 may have the same pattern as the radiopaque localization array 100 attached to the removable backing 220. In other examples, the cutter 204 may cut all the way through the backing layer 206 of the multilayer structure 218 only around the outermost edges of the radiopaque localization array 100. In such examples, the removable backing 220 shares the same outer outline of the radiopaque localization array 100, but the removable backing 220 is otherwise uncut within the outer outline. In still other examples, the backing layer 206 of the multilayer structure 218 may be cut by the cutter 204 or some other cutter in locations that differ from where the radiopaque localization array 100 is cut. As a result, the removable backing 220 may extend beyond the outer outline of the radiopaque localization array 100 and/or be removably attached to multiple radiopaque localization arrays. In such examples, the radiopaque localization array 100 may be kiss cut in the radiopaque layer 202 of the multilayer structure 218.

In some examples, the cutter 204 cuts just one radiopaque localization array 100 with each cut. In other examples, the cutter 204 cuts multiple radiopaque localization arrays with each cut. In the example illustrated in FIG. 2, the method 200 includes arranging the radiopaque localization arrays in a stack 222.

The method 100 may be implemented with additional, different, or fewer steps than in the above examples. For example, the method may include removing any waste from the radiopaque localization array 100 remaining after the cut is made by the cutter 204.

As another example, combining the radiopaque layer 202 and the backing layer 206 with the adhesive layer 212 may comprise applying the adhesive layer 212 to a first side of the backing layer 206 and bringing the radiopaque layer 202 in contact with the adhesive layer 212. The first side of the backing layer 206 may be a side of the backing layer 206 that faces the radiopaque layer 202 as the two layers 206 and 202 are combined. Alternatively, or in addition, combining the radiopaque layer 202 and the backing layer 206 with the adhesive layer 212 may comprise applying the adhesive layer 212 to a first side of the radiopaque layer 202 and bringing the adhesive layer 212 in contact with the backing layer 206. The first side of the radiopaque layer 202 may be the side of the radiopaque layer 202 that faces the backing layer 202 as the two layers 206 and 202 are combined. Applying the adhesive layer 212 to the first side of radiopaque layer 202 and/or to the first side of the backing layer 206 may include spraying or otherwise applying an adhesive to the first side of radiopaque layer 202 and/or to the first side of the backing layer 206. In some examples, spraying or otherwise applying an adhesive to the first side of radiopaque layer 202 and/or to the first side of the backing layer 206 may be in addition to or in lieu of feeding the adhesive layer 212 between the radiopaque layer 202 and the backing layer 206.

In the example illustrated in FIG. 2, the radiopaque layer 202, the backing layer 206, and the adhesive layer 212 are provided by feeding the radiopaque layer 202, the backing layer 206, and the adhesive layer 212 from spools 208, 210, and 214. In alternative examples, the radiopaque layer 202, the backing layer 206, and/or the adhesive layer 212 may be provided by feeding a sheet of the radiopaque layer 202, the backing layer 206, and/or the adhesive layer 212, respectively.

The radiopaque layer 202, the backing layer 206, and/or the adhesive layer 212 may include organic fiber cloth or yarn, metal, and/or one or more polymers or adhesives. Examples of the organic fiber cloth include an organic yarn, cotton, an/or any other cloth material. The radiopaque layer 202, the backing layer 206, the adhesive layer 212, and or any other layer included in the multilayer structure 218 may comprise one or more radiation attenuating, shielding and/or blocking substances in all or in a portion of the respective layer.

The adhesive layer 212 or any other layer, such as the radiopaque layer 202, may comprise, and/or be treated with, a substance with antimicrobial, anticolonization, antiviral, fungicidal or anti-yeast properties. In some examples, the radiopaque layer 202, the multilayer structure 218, and/or any layer therein may be sterilized.

The radiopaque layer 202, the multilayer structure 218, any layer therein, and/or any portion of the aforementioned layer(s) may be transparent to visible light and/or be opaque to visible light. The transparent layer and/or transparent portion may enable a health care practitioner to see the surface 104 of the patient.

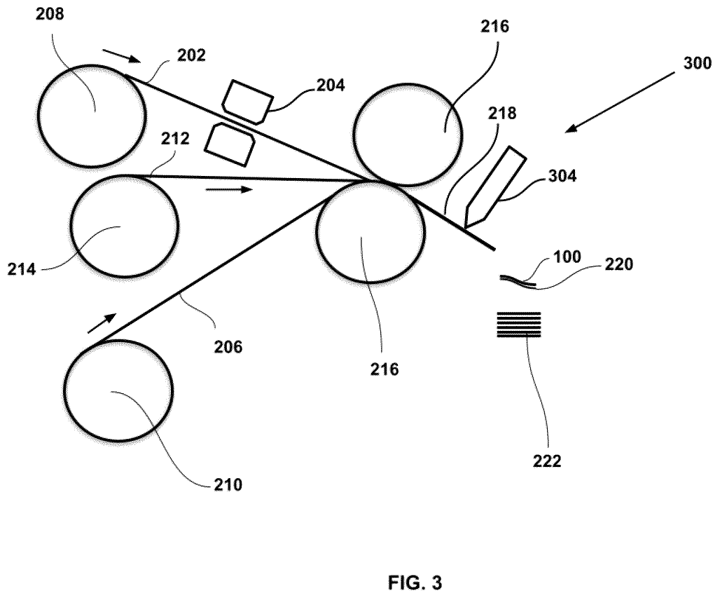

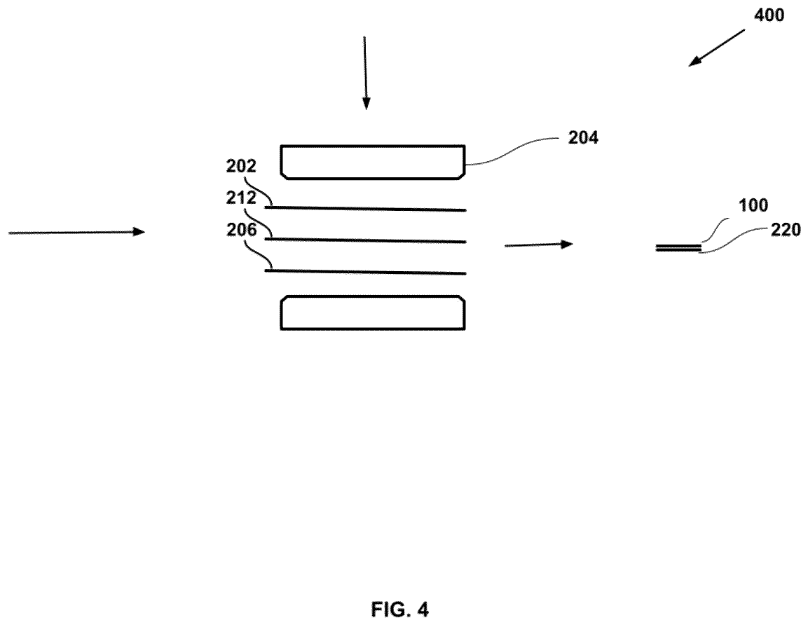

The steps in the method 100 may be implemented in different orders than described above. In the example illustrated in FIG. 2, the radiopaque location array 100 is cut in the radiopaque layer 202 after combining the radiopaque layer 202 and the backing layer 206. In an alternative example illustrated in FIG. 3, the radiopaque location array 100 is cut in the radiopaque layer 202 by the cutter 204 before combining the radiopaque layer 202 and the backing layer 206. However, all edges of the radiopaque location array 100 may not necessarily be cut by the cutter 204. For example, a leading and a trailing edge of the radiopaque location array 100 may be cut instead by a second cutter 304 so that the radiopaque layer 202 may still be fed to the two rollers 216 where the radiopaque layer 202 and the backing layer 206 are combined. In yet another example, the radiopaque location array 100 is cut in the radiopaque layer 202 when combining the radiopaque layer 202 and the backing layer 206. In one such an example, the cutter 204 may be a rotary die cutter which is positioned in the same location as—and takes the place of—any of the two rollers 216 shown in FIG. 2. In another such example, which is shown in FIG. 4, the cutter 204 may be a die cut machine that both presses the radiopaque layer 202, the adhesive layer 212, and the backing layer 206 together and cuts the radiopaque location array 100 in the radiopaque layer 202. In the example shown in FIG. 4, the radiopaque layer 202, the adhesive layer 212, and the backing layer 206 are provided as sheets. Only one radiopaque location array 100 is shown being cut by the cutter 204, however, the cutter 204 may cut multiple radiopaque location arrays 100 at once from the sheet of the radiopaque layer 202.

In FIG. 2, the radiopaque location array 100 is attached to the removable backing 220 by the adhesive layer 212. In an alternative example, the radiopaque location array 100 may not be attached to, or used with, any other layer. In still other examples, the radiopaque location array 110 may be attached to and/or used with another material and/or layer. For example, the radiopaque location array 110 may be attached to, and/or used with, a drape, an incise drape, tape, fabric, polymer, blanket, warming blanket, clothing, and/or a covering.

An incise drape may be placed on the patient's skin and cut through. Incise drapes may protect a wound from contamination or infection. Incise drapes may comprise, for example, a transparent polymer(s) and may further comprise an adhesive layer. The adhesive layer on the drape may comprise an antimicrobial agent in some examples. Some antimicrobial agents frequently used in incise drapes include chlorhexidine or betadine. The radiopaque location array 100 may comprise a portion of the incise drape. In some examples, the radiopaque location array 100 may comprise a removable portion of the incise drape such that removing the radiopaque components enables improved, higher quality, more legible, easier, faster visualization or interpretation of radio imaging.

The radiopaque location array 100 may be used individually, as part of an incise drape, in combination with a drape, in combination with a shield to reduce radiation scatter from a patient, and/or in combination with other garments, gowns, clothing gloves components used in an operating room to shield the patient or health care providers from fluids, blood, bacteria, or viruses.

The radiopaque location array 100 may be used in a system comprising two or more components, namely, the radiopaque location array 100, protective gowns, protective drapes, covers, clothing, gowns, scrubs and/or any other component configured to shield a person from contamination or infection by fluids, blood, bacteria or viruses or a component configured to attenuate radiation such as drapes, shields, attenuating clothing, covers, gowns, scrubs, and/or garments on which a distance from the source of radiation may be marked for reference by healthcare personnel. Distance from the source may be indicated by a measured tape or other measured markings. The system may also include a meter configured to measure a level of electromagnetic radiation emitted by the medical imaging device. When used together the components of the system may reduce the amount of radiation a person is exposed to and shield a person from exposure to contamination or infection from fluids, blood, bacteria, and viruses.

The grid component may be used as individually, as part of an incise drape, in combination with a drape, in combination with a shield to reduce radiation scatter from a patient, or in combination with other garments, gowns, clothing gloves components used in an operating room to shield the patient or health care providers from fluids, blood, bacteria, or viruses.

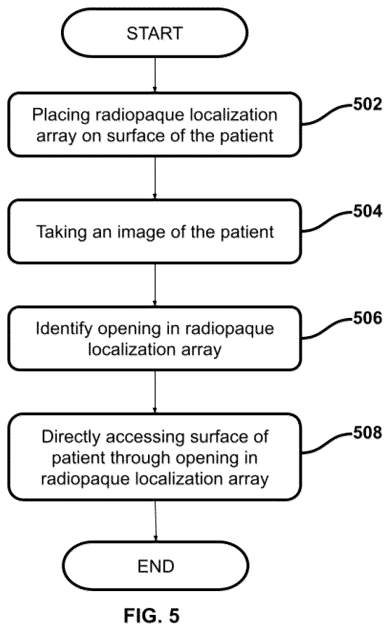

FIG. 5 illustrates an example of a method of using the radiopaque localization array 100 that leverages direct access to the surface 104 of the patient. The method may include additional, different, or fewer operations than illustrated in FIG. 5. Alternatively, or in addition, operations may be executed in a different order than illustrated in FIG. 5.

Operations may begin by a person, such as a health care practitioner, placing (502) the radiopaque localization array 100 on the surface 104 of the patient. In some examples, the adhesive layer 206 on the radiopaque localization array 100 may enable the radiopaque localization array 100 to attach to the surface 104 of the patient.

Operations may continue by the medical imaging machine taking (504) an image of the patient such that the radiopaque localization array 100 is superimposed on one or more internal features of the patient in the image taken by the medical imaging machine.

The person, such as a health care practitioner, may identify (506) an opening in the radiopaque localization array 100 in which to make an incision and/or an injection based on the markings 106 of the radiopaque localization array 100 that effectively identifies where a target internal feature of the patient is located. For example, a vertebra may be the target internal feature and the opening in which the vertebra appears in the image is the identified opening. In some examples, the person may identify (506) the opening in the radiopaque localization array 100 in which to make the incision and/or the injection based only on the markings 106 of the radiopaque localization array 100, and not on any additional object positioned in the radiopaque localization array 100 when the image of the patient was taken.

Operations may end, for example, by directly accessing (508) the surface 104 of the patient through the previously identified opening. For example, the person may directly access the surface 104 by making a mark with a writing instrument on, inserting a needle into, and/or making an incision in the surface 104 of the patient through the identified opening.

To clarify the use of and to hereby provide notice to the public, the phrases “at least one of <A>, <B>, . . . and <N>” or “at least one of <A>, <B>, . . . or <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

While various embodiments have been described, it will be apparent to those of ordinary skill in the art that many more embodiments and implementations are possible. Accordingly, the embodiments described herein are examples, not the only possible embodiments and implementations.

What is claimed is:

1. A method of manufacturing a radiopaque localization array comprising:
providing a radiopaque layer;
providing a backing layer;
combining the radiopaque layer and the backing layer with an adhesive layer between the radiopaque layer and the backing layer; and
cutting the radiopaque localization array in the radiopaque layer using a cutter.

2. The method of claim 1, wherein the radiopaque layer includes a metal foil.

3. The method of claim 1, wherein the radiopaque location array is cut in the radiopaque layer prior to combining the radiopaque layer and the backing layer.

4. The method of claim 1, wherein the radiopaque location array is cut in the radiopaque layer after combining the radiopaque layer and the backing layer.

5. The method of claim 1, wherein the radiopaque location array is cut in the radiopaque layer when combining the radiopaque layer and the backing layer.

6. The method of claim 1, wherein combining the radiopaque layer and the backing layer with the adhesive layer comprises feeding the adhesive layer between the radiopaque layer and the backing layer.

7. The method of claim 1, wherein the adhesive layer comprises a two-sided adhesive polymer.

8. The method of claim 1, wherein combining the radiopaque layer and the backing layer with the adhesive layer comprises applying the adhesive layer to a first side of the backing layer and bringing the radiopaque layer in contact with the adhesive layer.

9. The method of claim 1, wherein combining the radiopaque layer and the backing layer with the adhesive layer comprises applying the adhesive layer to a first side of the radiopaque layer and bringing the adhesive layer in contact with the backing layer.

10. The method of claim 1, wherein the radiopaque localization array comprises a metal grid that is attached by the adhesive layer to a removable backing.

11. The method of claim 1, wherein the cutter includes a die cut machine.

12. The method of claim 1, wherein the cutter includes a laser.

13. The method of claim 1, wherein the cutter includes a stamping press.

14. A method of manufacturing a radiopaque localization array comprising:
providing a radiopaque layer comprising a metal foil;
providing a backing layer;
combining the radiopaque layer and the backing layer with an adhesive layer between the radiopaque layer and the backing layer; and
cutting the radiopaque localization array in the radiopaque layer using a cutter after the radiopaque layer and the backing layer are combined, wherein the radiopaque localization array comprises a metal grid that is attached by the adhesive layer to a removable backing formed from the backing layer.

15. A method of using a radiopaque localization array, the method comprising:
placing the radiopaque localization array on a surface of a patient;
taking an image of the patient by a medical imaging machine, wherein the radiopaque localization array is superimposed on one or more internal features of the patient in an image taken by the medical imaging machine;
identifying an opening in the radiopaque localization array in which to make a mark on, an incision in, and/or an injection in based on a marking of the radiopaque localization array that identifies where a target internal feature of the patient is located; and
accessing the surface of the patient directly through the identified opening.

16. The method of claim 15, wherein the opening in the radiopaque localization array in which to make the mark, the incision, and/or the injection is identified based only on the markings of the radiopaque localization array, and not on any additional object positioned in the radiopaque localization array when the image of the patient was taken.
